(19) United States
(12) Patent Application Publication
(10) Pub. No.: US 2012/0020933 A1
(43) Pub. Date: Jan. 26, 2012

(54) METHOD OF NERVE REPAIR WITH AMNION AND CHORION CONSTRUCTS

(75) Inventors: Robin R. YOUNG, Wayne, PA (US); Richard M. JAY, Philadelphia, PA (US)

(73) Assignee: AFCELL MEDICAL, Parsippany, NJ (US)

(21) Appl. No.: 13/179,966

(22) Filed: Jul. 11, 2011

Related U.S. Application Data
(60) Provisional application No. 61/366,631, filed on Jul. 22, 2010.

(51) Int. Cl. A61K 35/48; A61B 19/02; A61P 25/00; A61F 2/02

(52) U.S. Cl. 424/93.7; 623/23.72; 206/438

(57) ABSTRACT

Improved methods for the surgical repair of a damaged or severed nerve are described. The improvement includes covering the surgically repaired nerve or a damaged neurilemma with at least one of an amniotic fluid and a construct, or replacing the damaged neurilemma with the construct prior to wound closing during the surgery. The construct contains an allograft having at least one layer of amnion and chorion tissues, and a shape adapted for enclosing a damaged or severed nerve or for covering or replacing the damaged neurilemma during the surgical repair. The products and methods improve the performance of the nerve surgery, e.g., by reducing inflammation, inhibiting fibrosis, scarring, fibroblast proliferation, post-operative infection while also promoting more rapid healing of damaged or traumatized nerve structures.

### CROSS-REFERENCE TO RELATED APPLICATIONS

This application is entitled to priority pursuant to 35 U.S.C. §119(e) to U.S. Provisional Patent Application No. 61/366,631, filed Jul. 22, 2010 which is hereby incorporated by reference herein in its entirety.

### BACKGROUND OF THE INVENTION

1. Field of Invention

Embodiments of the present invention relate to methods and products for improving the treatment of damaged nerves. In particular, embodiments of the present invention relate to constructs comprising an allograft patch having at least one of amnion tissue and chorion tissue for use during surgical repair of damaged or severed nerves to reduce inflammation, inhibit fibrosis, scarring, fibroblast proliferation, post-operative infection while also promoting more rapid healing of damaged or traumatized nerve structures.

2. Background of the Invention

Damage to nerves, whether peripheral nerves or nerves of the autonomic system, results in an extensive list of human maladies including, but not limited to, chronic pain, paralysis, impotence and absence of one or more of the five senses.

Repairing damaged nerves is hampered by the several factors including damage to the protective sheathing that surrounds nerves, gaps between the proximal and distal stumps of severed nerves and the limited ability of damaged nerves to regenerate. Most commonly, surgeons attempting to repair damaged or severed nerves use an autograft of a peripheral nerve harvested from the patient, e.g., a sural nerve autograft, sutures to bring the ends of the nerves together and then a nerve wrap in the form of a tube or conduit to protect the healed nerve. Despite numerous advances in surgical technique for repair of damaged nerves, the rate of success, according to recently published clinical studies, is poor with reported success rates ranging from 0% for severely paralyzed patients, to 13% for patients with peroneal nerve damage, to 85% for patient with radial nerve damage.

Furthermore, the tubes or conduits for guiding peripheral nerve regeneration are commonly made of materials such as polylactide, polylactide/polyglycolide copolymers, acrylic copolymers, polyvinylidene fluoride, polyglactin mesh, Millipore filter material, silicone, GORE-TEX®, arterial cuffs, performed mesothelial tubes or various other synthetic polyesters. The shortcomings of using a tube or conduit made of these materials include, for example, immune responses, induction of scar tissue, difficulty in application and development of local elevated concentrations of compounds released after degradation of a degradable material used in the device.

Therefore, it would be advantageous to have an alternative nerve sheathing material that would effectively inhibit fibroblast formation, scarring and adhesion formation while also promoting the natural healing process of a damaged or severed nerve. Currently there is no allograft product available for use during surgical repair of damaged or severed nerves which would promote healing while also reducing inflammation, scarring and adhesions.

Human nerves are covered by an extensive network of sheathing membranes. This protective network, generally called fascia, extends from the head to foot, front to back of the human body and covers all internal structures. Depending on the structure, fascia may be thick or thin and performs several critical functions to maintain health of internal organs, nerves, bones and nerves.

The protective sheathing surrounding nerves sheath consists of concentric layers of myelin which not only allow nerves to exist safely beside adjacent structures but also forms an electrical insulator that serves to speed the conduction of nerve impulses. This sheathing is also referred to as the sheath of Schwann or neurilemma. Loss or disruption of the neurilemma causes harm to the nerve and significantly limits the ability of damaged or severed nerves to heal.

A form of fascia that is similar to the neurilemma is created during pregnancy to protect and facilitate the development of a fetus. This membrane is the amnion and its adjacent membrane, the chorion. These two inner linings of the placental sac surround and protect embryos in reptiles, birds, and mammals in a form of sheathing that is similar to the fascia sheathing that protects all other internal structures of the human body including nerves.

The placental sac contains the fetus and amniotic fluid or liquor amnii, in which the embryo is immersed, nourished and protected. The two layers of amnion and chorion are tough, transparent, nerve-free, and nonvascular membranes consisting of multiple layers of cells: an inner, thick layer of ectodermal epithelium and an outer covering of mesodermal, connective, and specialized smooth muscular tissue. In the later stages of pregnancy, the amnion expands to come in contact with the inner wall of the chorion creating the appearance of a thin wall of the sac extending from the margin of the placenta. The amnion and chorion are closely applied, though not fused, to one another and to the wall of the uterus. Thus, at the later stage of gestation, the fetal membranes are composed of two principal layers: the outer chorion that is in contact with maternal cells and the inner amnion that is bathed by amniotic fluid.

The amnion has multiple functions, e.g., as a covering epithelium, as an active secretary epithelium, and for intense intercellular and transcellular transport. Before or during labor, the sac breaks and the fluid drains out. Typically, the remnants of the sac membranes are observed as the white fringe lining the inner cavity of the placenta expelled after birth. The amnion can be stripped off from the placenta. The amnion has a basement membrane side and a stroma side.

The fetal membrane including amnion and chorion has been used in surgeries documented as early as 1910. See Trelford et al., 1979, Am J Obstet Gynecol, 134:833-845. Amnioplastin, an isolated and chemically processed amniotic membrane, was used for continual dural repair, peripheral nerve injuries, conjunctival graft and flexor and tendon repair. See e.g., Chao et al., 1940, The British Medical Journal, March 30. The amnion has been used for multiple medical purposes, e.g., as a graft in surgical reconstruction forming artificial vaginas or over the surgical defect of total glossectomy, as a dressing for burns, on full-thickness skin wounds or in omphalocele, and in the prevention of meningocerebral adhesions following head injury or tissue adhesion in abdominal and pelvic surgery.

In recent years, there have been renewed interests in the application of amnion in ocular surface reconstruction, for example, as an allograph for repairing corneal defects. See, for example, Tsai and Tseng, Cornea. 1994 September; 13(5):389-400; and Dua et al., Br. J. Ophthalmol 1999, 83:748-20 752. In addition, amnion and amniotic fluid have recently been used as sources of placental stem cells. See, e.g., U.S. Pat. No. 7,255,879 and WO 200073421.

Despite the clinical and published record regarding the safety and efficacy of amnion in broad surgical use, issues regarding reproducibility, safety and the precise form of amnion for each prospective indication have prevented amnion from achieving broad commercial distribution.

There is a need of improved methods and products for improved surgical repair of damaged or severed nerves to reduce inflammation, inhibit fibrosis, scarring, fibroblast proliferation, post-operative infection while also promoting more rapid healing of damaged or traumatized nerve structures. The present invention relates to such improved methods and products.

### BRIEF SUMMARY OF THE INVENTION

In one general aspect, embodiments of the present invention relate to a construct for use in surgical repair of a damaged or severed nerve, the construct comprising an allograft comprising at least one layer of human amnion and chorion tissues, wherein the construct has a shape adapted for enclosing the damaged or severed nerve or for covering or replacing a damaged neurilemma during the surgical repair.

In another general aspect, embodiments of the present invention relate to a method of preparing a construct for use in surgical repair of a damaged or severed nerve, the method comprising drying an allograft comprising at least one layer of human amnion and chorion tissues on a frame, wherein the construct has a shape adapted for enclosing the damaged or severed nerve or for covering or replacing a damaged neurilemma during the surgical repair. In one embodiment of the present invention, the frame is rigid, semi-rigid or flexible.

Another aspect of the present invention relates to a method of performing a surgical repair of a damaged or severed nerve in a subject, comprising:

(a) surgically repairing the damaged or severed nerve to obtain a surgically repaired nerve in the subject; and

(b) covering the surgically repaired nerve or a damaged neurilemma with at least one of an amniotic fluid and a construct, or replacing the damaged neurilemma with the construct prior to wound closing,

wherein the construct comprises at least one layer of human amnion and chorion tissues, and the construct has a shape appropriate for enclosing the surgically repaired nerve or for covering or replacing the damaged neurilemma.

Yet another general aspect of the present invention relates to a kit, which comprises:

(a) at least one of an amniotic fluid and a construct for use in surgical repair of a damaged or severed nerve; and

(b) instructions on how to use the amniotic fluid and the construct in the surgical repair,

wherein the construct comprises an allograft comprising at least one layer of amnion and chorion tissues, wherein the construct is adapted for enclosing the damaged or severed nerve or for covering or replacing a damaged neurilemma during the surgical repair.

In a preferred embodiment of the present invention, the human amnion and chorion tissues used in the present invention are obtained by a process comprising:

(a) obtaining informed consent from pregnant females;

(b) conducting risk assessment on the consented pregnant females to select an amnion donor;

(c) procuring after birth placenta from the amnion donor; and

(d) obtaining the human amnion and chorion tissues from the placenta.

Other aspects, features and advantages of the invention will be apparent from the following disclosure, including the detailed description of the invention and its preferred embodiments and the appended claims.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown.

In the drawings:

FIG. 1 illustrates the structural arrangement of the nerve and nerve sheath;

FIG. 2 illustrates an example of a nerve surgery, i.e., epineural repair of damaged or severed nerves;

FIG. 3 illustrates an embodiment of the present invention where a construct of amnion and chorion tissues is applied to the damaged or severed nerves during a nerve surgery;

FIG. 4 illustrates constructs for use in a surgical repair of a damaged or severed nerve of various shapes according to embodiments of the present invention; and

FIG. 5 illustrates the structure and composition of amnion and chorion.

### DETAILED DESCRIPTION OF THE INVENTION

Unless defined otherwise, all technical and scientific terms used herein have the same meaning as commonly understood to one of ordinary skill in the art to which this invention pertains.

In this application, certain terms are used, which shall have the meanings as set in the specification. It must be noted that as used herein and in the appended claims, the singular forms “a,” “an,” and “the” include plural reference unless the context clearly dictates otherwise.

Embodiments of the present invention relate to an amnion and/or chorion allograft construct for use in a nerve repair surgery. The construct comprises at least one layer of amnion and chorion tissues, preferably the amnion and chorion tissues are from human. The construct is made into a shape and thickness uniquely designed for enclosing a damaged or severed nerve or for covering or replacing a damaged neurilemma during the surgical repair. The construct can be made by drying an allograft patch of amnion and/or chorion membranes into the required shape or over a resorbable frame, e.g., polymer mesh frame, or a disposable or stainless steel frame. The frame can be flexible, rigid or semi-rigid. The configuration of the construct allows for ease of application in a nerve surgery to thereby facilitate and improve surgical repair of damaged or severed nerves. The construct can further comprise one or more active agents that facilitate the surgical repair. The active agents include, but are not limited to, anti-microbial agents, growth enhancing agents, anti-inflammatory agents, analgesics, etc.

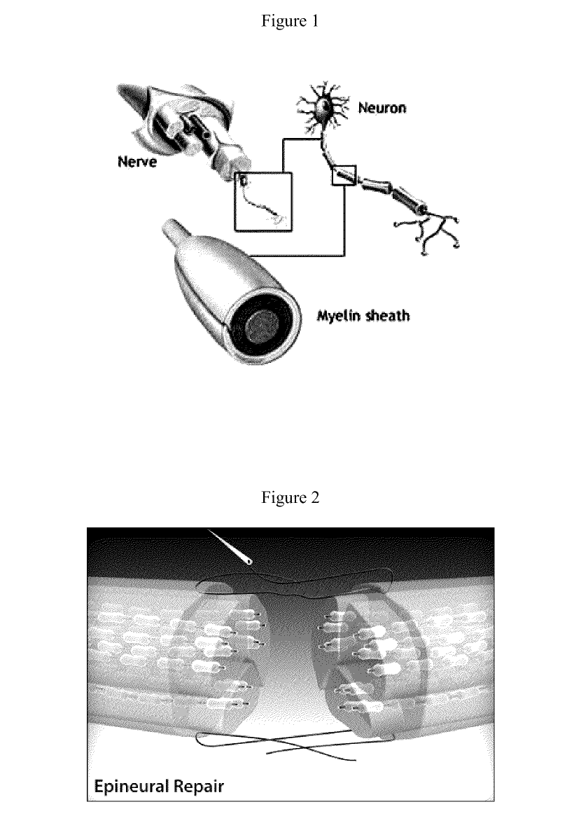

Each nerve fiber consists of a number of nerve fibrils collected into a central bundle, the axis cylinder, which is surrounded by neurolemma (also known as neurilemma or sheath of Schwann), a thin membrane. Between the neurolemma and the axis cylinder is myelin sheath, a electrically insulating fatty substance. Both the neurolemma and the myelin sheath are insulating agents that are essential for the proper functioning of the nervous system (FIG. 1).

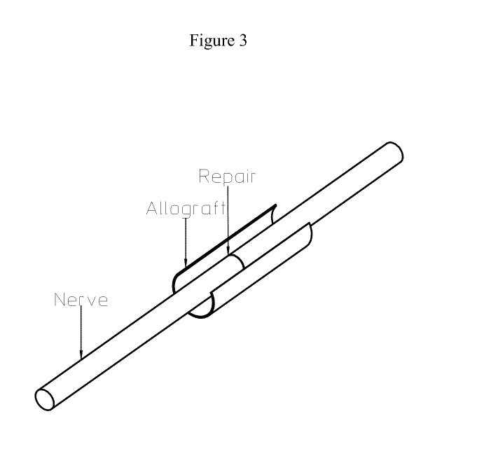

Surgeries are required to repair certain nerve conditions, such as entrapment (carpal tunnel disease, cubital tunnel disease, peroneal nerve entrapment), tumors or trauma in central or peripheral nerve system. For example, epineural nerve repair has been used to repair injuries to sensory or pure motor nerves or in case a peripheral nerve has become cut or otherwise divided. In the surgery, the injured nerve is identified and exposed so that normal nerve tissue can be examined above and below the level of injury, usually with magnification, using either loupes or an operating microscope. If a large segment of nerve is harmed, as resulting from a crush or stretch injury, the nerve will need to be exposed over a larger area. Injured portions of the nerve are removed. The cut nerve endings are then carefully reapproximated using very small sutures, see e.g., FIG. 2. The nerve repair must be covered by healthy tissue. In traditional epineural nerve repair, the covering step can be simply closing the skin or using autograft skin or muscle moved from other parts of the body to provide healthy padded coverage over the nerve.

Another general aspect of the present invention relates to a method of performing a surgical repair of a damaged or severed nerve in a subject. The method comprises: (a) surgically repairing the damaged or severed nerve to obtain a surgically repaired nerve in the subject; and (b) covering the surgically repaired nerve or a damaged neurilemma with at least one of an amniotic fluid and a construct, or replacing the damaged neurilemma with the construct prior to wound closing, wherein the construct comprises at least one layer of human amnion and chorion tissues, and the construct has a shape appropriate for enclosing the surgically repaired nerve or for covering or replacing the damaged neurilemma.

The amniotic fluid and the construct can be applied individually or in combination during the surgery. The damaged neurilemma can be associated with the damaged or severed nerve. The damaged neurilemma can also be resulting from the surgical repair of the damaged or severed nerve. Preferably, the amniotic fluid is processed so that it has a relatively high viscosity for ease of application and for remaining in the desired area after the application.

In one embodiment of the present invention, both the amniotic fluid and the replacement cover are applied during the surgery, preferably, the amniotic fluid has a relatively high viscosity.

In another embodiment of the present invention, only the amniotic fluid is applied to cover the surgically repaired nerve or the damaged neurilemma, preferably the amniotic fluid has a relatively high viscosity.

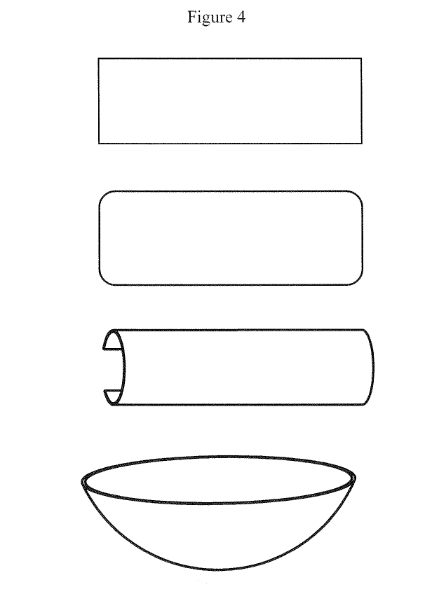

In a preferred embodiment of the present invention, a construct according to an embodiment of the present invention that comprises an allograft patch having at least one layer of amnion and chorion tissues is used to enclose a damaged or severed nerve or to cover or replace the damaged neurilemma during a surgical repair, see e.g., FIG. 3. The construct is of a shape suitable for enclosing the damaged or severed nerve or for covering or replacing the damaged neurilemma during the surgical repair, including, but not limited to, flat sheets, cylindrical shapes, generally cylindrical shapes with a C-shaped cross-section, concave bowls or curved sheets, see e.g., FIG. 4.

In one embodiment of the present invention, the allograft construct of amnion and/or chorion is processed in such a way as to become a rigid or semi-rigid shape which is appropriate for the surgical need. Such shapes can be tubular, concave bowls or curved sheets, as examples. Whichever form is used, the resulting allograft construct retains its shape up to and including implantation into the patient. Following implantation and when the tissue is hydrated, the shape can relax and the implanted membrane can conform to the nerve tissues. The shapes can stop short of touching end to end to allow for ease of implantation over the nerve.

In one embodiment of the present invention, the construct further comprises a frame, which can be rigid, semi rigid or flexible, preferably the frame is rigid and semi rigid. The frame can be disposable or implantable and resorbable.

In another embodiment of the present invention, one or more corners of the construct are rounded or flatted to prevent the corners from catching during implantation. In view of the present disclosure, any method known to those skilled in the art can be used to make the corners of the construct round or flatten.

The construct can be of various lengths and diameters to fit the various nerve structures in the body. For example, the thickness of the allograft comprising amnion and/or chorion can be between 0.25 mm to 2.0 mm.

In one embodiment of the present invention, the allograft construct is tubular or cylindrical having a first end and a second end, a wall of a uniform thickness and an open end to allow for simple implantation over the nerve structure.

In another embodiment of the present invention, a generally cylindrical allograft construct is used in a surgical repair of an injured peripheral nerve. The allograft construct can be applied around the nerve for indications such as: crush injuries, stretch injuries, nerves that have been partially severed, nerve injuries and nerve repairs that require separation and protection from surrounding soft tissues, compression injuries, over direct suture repair, penetrating injury, lacerations/stabbing injuries, repair of iatrogenic nerve injury, and failed primary repair.

According to an embodiment of the present invention, a cylindrical allograft is indicated in the management of peripheral nerve injuries. The typical sizes of the cylindrical allograft can be 3 mm and 5 mm in diameter and 30 mm in length. With Loupe magnification, an incision is created over the injured nerve. The nerve is located proximally and distally from the injured site. Neurolysis is performed, freeing it from the surrounding scar tissue. The nerve is mobilized. Using a Penrose drain or a blunt retraction the nerve is delivered and prepared for the cylindrical allograft. The nerve is measured to allow proper sizing of the selected cylindrical allograft. Gently separate the amniotic cylindrical allograft around the injured nerve. The cylindrical allograft is then released and will rest directly around and in contact with the nerve. The cylindrical allograft will adhere to the nerve. Optionally, the cylindrical allograft can be sutured especially if a gliding mechanism is present using #6.0 to #8.0 atraumatic absorbable suture tacking the cylindrical allograft to the epineurium at the most distal and proximal ends away from the injured neural site. Excess cylindrical allograft can be removed prior to hydration. Hydrate neural amniotic cylindrical allograft with sterile saline. The surgical field is then closed in layers over the cylindrical allograft nerve.

In another embodiment of the present invention, a construct comprising a layer of amnion is used to cover a skin incision resulting from the surgery. The allograft patch can be of any size suitable for covering the sutures or other type of tissue injuries at skin incision.

Preferably, a relatively thick layer of allograft is used to cover the skin incision. In one embodiment of the invention, the allograft patch has a thickness of about 2 mm to 4 mm. It can have multiple layers of amnion or a combination of multiple layers of amnion and chorion in any combination of amnion and chorion.

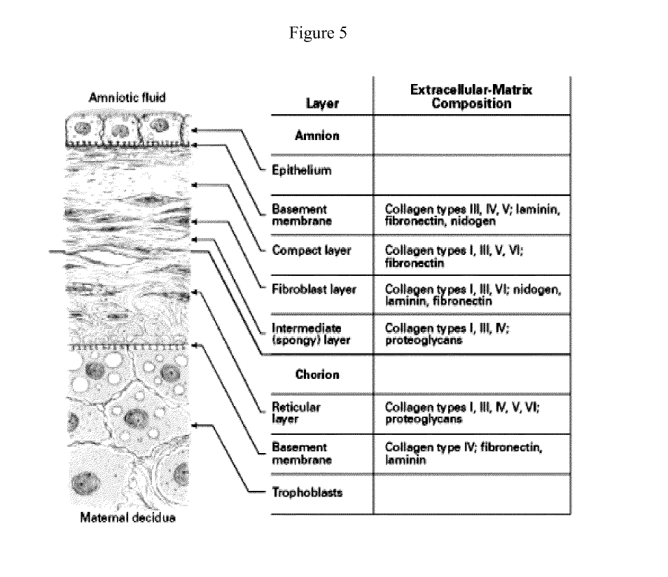

There are several attributes which make Amnion a preferred material for protecting nerves which have been surgically repaired with sutures or other connective devices. FIG. 5 illustrates the structure and composition of amnion and chorion. Amnion has a complete lack of surface antigens and therefore does not induce an immune response when implanted into a ‘foreign’ body, which is in contrast to most other implants. Amnion also markedly suppresses the expression of the pro-inflammatory cytokines, IL-1α and IL-1β (Solomon et al., 2001, Br J Ophthalmol. 85(4):444-9) and produces natural inhibitors of matrix 20 metalloproteases (MMPs) expressed by infiltrating polymorphonuclear cells and macrophages. Hao et al., 2000, Cornea, 19(3):348-52; Kim et al., 2000, Exp Eye Res. 70(3):329-37). Amnion also down-regulates TGF-β and its receptor expression by fibroblasts leading to the ability to modulate the healing of a wound by promoting tissue reconstruction. Furthermore, amnion and chorion contain antimicrobial compounds with broad spectrum activity against bacteria, fungi, protozoa, and viruses for reduced risk of post-operative infection. All of these characteristics of amnion make it suitable for use in treating damaged or severed nerves.

Human allograft amnion and chorion have the ability to prevent scarring, reduce inflammation, inhibit microbial infection and improve healing. Repairing torn nerves, however, requires the surgeon to work in very tight spaces and repairing the nerve sheath is extremely difficult. Surgeons who would attempt to repair the nerve sheath with a replacement membrane could encounter several problems. Curving a flat sheet around a small nerve at the surgical site is extremely difficult for the surgeon.

By creating a rigid, semi-rigid or flexible curved shape which mimics the size and characteristics of a human nerve the present invention improves the ability of the surgeon to reduce adhesions, scar formation while also reducing inflammation and risk of post-operative infection following surgical repair of nerves.

Amnion tissues used in the present invention can be prepared from birth tissue procured from a pregnant female. Informed consent is obtained from a pregnant female by following guidelines as promulgated by the American Association of Tissue Banks and consistent with guidelines provided the Food and Drug Administration: a federal agency in the Department of Health and Human Services established to regulate the release of new medical products and, finally, if required by an established review body of the participating hospitals or institutions. The pregnant female is informed that she will be subject to risk assessment to determine if she is qualified as a birth tissue donor. She will also be informed of the tests for the risk assessment. The pregnant female is further informed that, if she is selected as a birth tissue donor based on the risk assessment, her birth tissues, such as placenta and amniotic fluid, may be collected at birth, tested and processed for medical uses.

The informed consent includes consent for risk assessment and consent for donation of birth tissues.

Risk assessment is conducted on a pregnant female with informed consent to evaluate her risk factors for communicable diseases, such as human immunodeficiency virus (HIV), hepatitis B virus (HBV), hepatitis C virus (HCV), cytomegalovirus (CMV), human T-lymphotropic virus (HTLV), syphilis, etc. Medical and social histories of the pregnant female, including physical exam record, and/or risk assessment questionnaire, are reviewed. Pregnant females with high risk factors for the communicable diseases are excluded.

Consent to draw blood at time of delivery and 1 to 12 months post delivery is obtained from pregnant females with low risk factors for the communicable diseases. Screening tests on communicable diseases, such as HIV 1 and 2, HCV, HbCore, syphilis, HTLV I/II, CMV, hepatitis B and C, are conducted by conventional serological tests on the blood sample obtained at birth. The initial screening tests are preferably completed within 7 days after birth. Preferably, the screening tests are conducted again on a second blood sample collected a few months post delivery, to verify the previous screening results and to allow for detection of communicable disease acquired shortly before birth, but are shown as “negative” on the previous screening tests. The second blood sample can be collected 1-12 months, preferably 6 months, post birth.

Only pregnant females with informed consent who are tested negative for the communicable diseases are approved as birth tissue donor. In a preferred embodiment, only pregnant females with informed consent who are tested negative for the communicable diseases in both screening tests with the blood sample drawn at birth and the blood sample drawn 6 months post delivery are approved as birth tissue donor.

Sterile techniques and procedures should be used as much as practically possible in tissue handling, e.g., during tissue procurement, banking, transfer, etc., to prevent contamination of the collected tissues by exogenous pathogens.

Only birth tissues procured from the approved birth tissue donors are subject to the collection and subsequent processing. Birth tissues, such as placenta and amniotic fluid, are recovered from the delivery room and are transferred to a location in a sterile container, such as a sterile plastic bag or bottle. Preferably, the tissues are transferred in a thermally insulated device at a temperature of 4° to 28° C., for example, in an ice bucket.

According to an embodiment of the invention, shortly after its expulsion after birth, a suitable human placenta is placed in a sterile zip-lock plastic bag, which is placed in an ice bucket, and is delivered to another location. The placenta is rinsed, e.g., with sterile saline, to removed excessive blood clots. Preferably, the placenta is subject to aseptic processing, for example, by including one or more antibiotics, such as penicillin and/or streptomycin, in the rinse. The aseptically processed placenta is stored in a controlled environment, such as hypothermic conditions, to prevent or inhibit apoptosis and contamination.

The processed placenta is placed in a sterile container, such as one made of triple sterile plastic bags, packed in wet ice, and shipped to a location for subsequent processing via overnight courier. The placenta is shipped together with release documents for processing. For example, each shipment must include technical approval to process based upon a satisfactory review of the criteria for donor selection and donor approval. The shipment must also include results on screening of communicable diseases. Preferably, the shipment includes medical director review and approval of donor eligibility/suitability.

Upon receiving the shipment and a satisfactory review of the accompanying release documents, the amnion is separated from the chorion and other remaining tissues of placenta using methods known in the art in view of the present disclosure. For example, the amnion can be stripped off mechanically from the placenta immersed in an aseptic solution, e.g., by tweezers. The isolated amnion can be stored in a cryoprotective solution comprising a cryoprotective agent, such as dimethyl sulfoxide (DMSO) and glycerol, and cryopreserved by using a rapid, flash-freeze method or by controlled rate-freeze methods. Preferably, the isolated amnion is treated with one or more antibiotics, such as penicillin and/or streptomycin, prior to cryopreservation. The chorion can also be separated from the other tissues, preserved and stored for future use.

The isolated amnion is a tough, transparent, nerve-free and nonvascular sheet of membrane. It can be dried or lyophilized using various methods. For example, it can be dried over a sterile mesh, for example, by being placed on a sterile nitrocellulose filter paper and air dried for more than 50 minutes in a sterile environment. It can also be dried or lyophilized over other form of supporting material, which would facilitate the subsequent manipulation of the amnion, such as sterilizing, sizing, cataloging, and shipping of the amnion.

The present invention encompasses a kit comprising at least one of an amniotic fluid and an allograft construct for use in a surgical repair of a damaged or severed nerve and instructions on how to use the amniotic fluid and the construct in the surgery. Any of the constructs for use in a nerve surgery according to embodiments of the present invention can be included in the kit. The construct comprises an allograft patch comprising at least one layer of human amnion and chorion tissues. The construct is adapted for enclosing the damaged or severed nerve or for covering or replacing a damaged neurilemma during the surgical repair. In a preferred embodiment, the kit comprises a plurality of constructs for the nerve surgery, and at least two of the plurality of constructs have different shapes or sizes suitable for covering different surgical sites. One or more corners of the construct can preferably be rounded or flatted to prevent the corner from catching during implantation. The allograft patch in the construct can further comprise one or more therapeutically active agents, such as anti-microbial agents, growth enhancing agents, anti-inflammatory agents, analgesics, etc.

It will be appreciated by those skilled in the art that changes could be made to the embodiments described above without departing from the broad inventive concept thereof It is understood, therefore, that this invention is not limited to the particular embodiments disclosed, but it is intended to cover modifications within the spirit and scope of the present invention as defined by the appended claims.

I/we claim:

1. A construct for use in a surgical repair of a damaged or severed nerve, the construct comprising an allograft comprising at least one layer of human amnion and chorion tissues, wherein the construct has a shape adapted for enclosing a damaged or severed nerve or for covering or replacing a damaged neurilemma during the surgical repair.

2. The construct of claim 1 further comprising a frame that is disposable or implantable and resorbable.

3. The construct of claim 2, wherein the frame is rigid or semi rigid.

4. The construct of claim 1, having a shape selected from the group consisting of a flat sheet, a cylindrical shape, a generally cylindrical shape with a C-shaped cross-section, a concave bowl and a curved sheet.

5. The construct of claim 1, having one or more rounded or flattened corners.

6. The construct of claim 1, further comprising one or more active agents.

7. The construct of claim 6, wherein the active agent is selected from the group consisting of anti-microbial agents, growth enhancing agents, anti-inflammatory agents, and analgesics.

8. The construct of claim 1, wherein the human amnion and chorion tissues are obtained using a process comprising:
a. obtaining informed consent from pregnant females;
b. conducting risk assessment on the consented pregnant females to select an amnion donor;
c. procuring after birth placenta from the amnion donor; and
d. obtaining the human amnion and chorion tissues from the placenta.

9. A method of preparing a construct of claim 2, the method comprising drying the allograft comprising at least one layer of human amnion and chorion tissues on the frame.

10. A method of performing a surgical repair of a damaged or severed nerve in a subject, comprising:
(a) surgically repairing the damaged or severed nerve to obtain a surgically repaired nerve in the subject; and
(b) covering the surgically repaired nerve or a damaged neurilemma with at least one of an amniotic fluid and a construct, or replacing the damaged neurilemma with the construct prior to wound closing,
wherein the construct comprises at least one layer of human amnion and chorion tissues, and the construct has a shape appropriate for enclosing the surgically repaired nerve or for covering or replacing the damaged neurilemma.

11. The method of claim 10, further comprising applying one or more allografts comprising at least one layer of human amnion and chorion tissues over one or more suture lines and incisions resulting from the surgical repair to form a cover and barrier over the suture lines and the incisions.

12. The method of claim 10, wherein the damaged or severed nerve is associated with an indication selected from the group consisting of crush injuries, stretch injuries, nerves that have been partially severed, nerve injuries and nerve repairs that require separation and protection from surrounding soft tissues, compression injuries, direct suture repair, penetrating injury, lacerations/stabbing injuries, iatrogenic nerve injury, and failed primary repair.

13. The method of claim 10, wherein the construct has a shape selected from the group consisting of flat sheets, cylindrical shapes, generally cylindrical shapes with a C-shaped cross-section, concave bowls and curved sheets.

14. The method of claim 10, wherein the construct has one or more rounded or flattened corners.

15. The method of claim 10, wherein the construct further comprises a frame that is disposable or implantable and resorbable.

16. The method of claim 10, wherein the construct comprises at least one layer of human amnion and at least one layer of chorion.

17. The method of claim 10, further comprising administering to the subject one or more additional treatments to the damaged or severed nerve selected from the group consisting of anti-microbial agents, growth enhancing agents, anti-inflammatory agents, and analgesics.

18. The method of claim 10, wherein the amniotic fluid and the human amnion and chorion tissues are obtained using a process comprising:
a. obtaining informed consent from pregnant females;
b. conducting risk assessment on the consented pregnant females to select an amnion donor;
c. procuring the amniotic fluid from the amnion donor;
d. procuring after birth placenta from the amnion donor; and
e. obtaining the human amnion and chorion tissues from the placenta.

19. A kit comprising:
(a) at least one of an amniotic fluid and a construct for use in surgical repair of a damaged or severed nerve; and
(b) instructions on how to use the amniotic fluid and the construct in the surgical repair,
wherein the construct comprises an allograft comprising at least one layer of amnion and chorion tissues, and the construct is adapted for enclosing the damaged or severed nerve or for covering or replacing a damaged neurilemma during the surgical repair.

20. The kit of claim 19, comprising a plurality of constructs for use in surgical repair of a damaged or severed nerve, wherein at least two of the plurality of constructs have different shapes or sizes.
